(19) United States
(12) Patent Application Publication
(10) Pub. No.: US 2026/0355070 A1
(43) Pub. Date: Nov. 12, 2026

(54) CORE-SHELL PARTICLE COMPRISING NATURAL POLYMER AND METHODS FOR PREPARING THE SAME

(71) Applicant: AMOREPACIFIC CORPORATION, Seoul (KR)

(72) Inventors: Najeeb CHOOLAKADAVIL KHALID, Yongin-si (KR); Byoung Young WOO, Yongin-si (KR); Bongsoo PI, Yongin-si (KR); Youngkyu HAN, Yongin-si (KR); Heungsoo BAEK, Yongin-si (KR); Sungyeon CHO, Yongin-si (KR)

(73) Assignee: AMOREPACIFIC CORPORATION, Seoul (KR)

(21) Appl. No.: 19/649,782

(22) Filed: Apr. 16, 2026

(30) Foreign Application Priority Data
May 9, 2025 (KR) 10-2025-0060215

(51) Int. Cl. A61K 8/02; A61K 8/25; A61K 8/27; A61K 8/29; A61K 8/73; A61Q 17/04; C09C 1/30

(52) U.S. Cl. CPC A61K 8/0241; A61K 8/25; A61K 8/27; A61K 8/29; A61K 8/732; A61K 8/735; A61Q 17/04; C09C 1/3054; C09C 1/3072; C09C 1/309; A61K 2800/614; A61K 2800/621; A61K 2800/63; A61K 2800/651; A61K 2800/85; C01P 2002/84; C01P 2004/03; C01P 2004/84

(57) ABSTRACT

One aspect of the present disclosure relates to core-shell particles comprising a natural polymer. More specifically, the core-shell particles according to one aspect may exhibit a form in which an inorganic powder is effectively coated on the surface of a core particle by including a predetermined natural polymer binder, ultimately, the core-shell particles, in which the inorganic powder is well-dispersed, are capable of exhibiting an excellent ultraviolet blocking effect.

### CROSS REFERENCE TO RELATED APPLICATION

The present application claims priority to Korean Patent Application No. 10-2025-0060215, filed May 9, 2025, the entire contents of which are hereby incorporated by this reference.

### BACKGROUND OF THE INVENTION

### Field of the Invention

The present disclosure relates to core-shell particles comprising a natural polymer and a method for manufacturing the same.

### Description of the Related Art

To prevent damage and aging of skin according to ultraviolet exposure, ultraviolet blocking agents are being widely used. In the ultraviolet blocking agents, there are organic ultraviolet blocking agents and inorganic ultraviolet blocking agents, and as the inorganic ultraviolet blocking agents, metal oxide powders such as ZnO and TiO₂ may be used. The metal oxide powders such as ZnO and TiO₂ have advantages that photostability is high and irritation is low compared to the organic ultraviolet blocking agents.

However, the metal oxide powders such as ZnO and TiO₂ are aggregated with each other by hydrophobic characteristics so that dispersibility within a formulation is lowered, and due to this, ultraviolet blocking efficacy may be reduced. In addition, aggregation of the metal oxide powders makes the metal oxide powders not to be uniformly applied to the skin, and there is a problem of deteriorating usability and aesthetics by inducing a white cast phenomenon.

To solve these problems, surface treatment methods using surfactants and the like have been attempted, but for these methods, treatment processes are complicated and there are limitations in an aspect of chemical stability. In addition, as preferences of consumers for natural ingredients have recently increased, demands for development of ultraviolet blocking agents utilizing naturally derived materials rather than chemical treatment methods are rising.

### SUMMARY OF THE INVENTION

One aspect of the present disclosure aims to provide a core-shell particle in which an inorganic powder is coated on a surface of core particles by using a natural polymer.

One aspect of the present disclosure aims to provide a composition for ultraviolet blocking which represents an excellent ultraviolet blocking effect and may be uniformly applied to skin while escaping a problem that an inorganic powder is aggregated.

One aspect of the present disclosure aims to provide a method for manufacturing a core-shell particle in which an inorganic powder is coated on a surface of core particles by using a natural polymer.

A core-shell particle according to one aspect of the present disclosure comprises a core comprising silica (SiO₂) particles; and a shell comprising an inorganic powder; and comprises a natural polymer binder for bonding the silica particles and the inorganic powder, wherein the natural polymer binder is at least one selected from the group consisting of hyaluronic acid and starch.

A core-shell particle according to one aspect of the present disclosure comprises a core and a shell, the core comprises silica particles surface-modified with a first natural polymer, and the shell comprises an inorganic powder surface-modified with a second natural polymer, and the first natural polymer and the second natural polymer are each independently at least one selected from the group consisting of hyaluronic acid and starch, and the first natural polymer and the second natural polymer are bonded.

A composition for ultraviolet blocking according to one aspect of the present disclosure comprises the core-shell particle according to one aspect.

A method for manufacturing a core-shell particle according to one aspect of the present disclosure comprises steps of: (a) manufacturing silica particles surface-modified with a first natural polymer; (b) manufacturing an inorganic powder surface-modified with a second natural polymer; and (c) manufacturing a core-shell particle by mixing the silica particles surface-modified with the first natural polymer and the inorganic powder surface-modified with the second natural polymer, wherein the first natural polymer and the second natural polymer are each independently at least one selected from the group consisting of i) high-pressure treated or enzyme treated hyaluronic acid and ii) high-pressure treated or enzyme treated starch.

According to one aspect of the present disclosure, an inorganic powder may be effectively coated on a surface of core particles by using a natural polymer binder which is high-pressure processed or enzyme treated, and as a result, a core-shell particle in which the inorganic powder is well dispersed and which represent an excellent ultraviolet blocking effect may be provided.

A core-shell particle according to one aspect of the present disclosure represents excellent dispersibility, and may be composed of OTC (Over-the-Counter) raw materials so that there is an advantage that additional permission may not be required as a raw material for an ultraviolet blocking agent. In addition, for the core-shell particle according to one aspect of the present disclosure, an inorganic powder is coated on a core particle surface, so that a scattering wavelength between ultraviolet-visible light may be controlled and a wavelength scattering intensity may be effectively increased to increase ultraviolet blocking power.

A composition comprising a core-shell particle according to one aspect of the present disclosure has excellent dispersibility of an inorganic powder, and may represent an excellent ultraviolet blocking effect (e.g., a light scattering effect, an SPF index, etc.) and an excellent feeling of use (e.g., being capable of being uniformly applied to skin), and water-dispersion formulation is possible.

A method for manufacturing a core-shell particle according to one aspect of the present disclosure has advantages that steps are simple (e.g., a simple mixing process of core particles and an inorganic powder), an excellent production yield may be represented, and almost no by-products are generated. In addition, a method for manufacturing a core-shell particle according to one aspect of the present disclosure may improve problems of conventional chemical methods for manufacturing a core-shell particle (e.g., a high manufacturing cost due to use of chemical substances, use of environmentally hazardous substances and emission problems in a manufacturing process).

### BRIEF DESCRIPTION OF THE DRAWINGS

FIG. 1 is an SEM image of SiO₂@ZnO core-shell particles of an Example according to one aspect.

FIG. 2 is an enlarged view of a part of an SEM image of SiO₂@ZnO core-shell particles of an Example according to one aspect.

FIG. 3 is an SEM image of SiO₂@TiO₂ core-shell particles of an Example according to one aspect.

FIG. 4 is SEM images of an Example using a natural polymer binder and a Comparative Example not using a natural polymer binder.

FIG. 5 is SEM images of an Example using a high-pressure treated natural polymer binder and a Comparative Example using a natural polymer binder which is not high-pressure treated.

FIG. 6 is UV-Vis absorption data results comparing light scattering effects of SiO₂@ZnO core-shell particles of an Example according to one aspect and an equivalent amount of ZnO.

FIG. 7 is photographs showing results of evaluating application uniformity of an Example and a Comparative Example according to one aspect.

FIG. 8 is SEM images of an Example using an enzyme treated natural polymer binder and a Comparative Example using a natural polymer binder which is not enzyme treated.

### DETAILED DESCRIPTION OF THE INVENTION

Various embodiments of this document and the terms used in the embodiments are not intended to limit the technical features disclosed in this document to the particular embodiments and should be understood as including various alterations, equivalents, or alternatives of the corresponding embodiments.

The inorganic powder, which was conventionally mainly used as an ultraviolet blocking agent, has a problem that it aggregates with each other due to hydrophobic characteristics and dispersibility is not good.

In one aspect, the present inventors have perceived that an inorganic powder may be effectively coated on a surface of core particles by using a predetermined natural polymer binder, and implemented core-shell particles in which the inorganic powder is well dispersed and an ultraviolet blocking effect is excellent. In one embodiment, a core-shell structure could be stably formed without an additional chemical reaction by modifying surfaces of SiO₂ particles and an inorganic powder with a predetermined natural polymer, respectively, and then mixing them.

### DEFINITION OF TERMS

In the present disclosure, the term “core-shell particles” may mean a composite structure including a core layer located in a center part and a shell layer surrounding a part or an entirety of the core layer. Here, “core” represents the center part of the core-shell particles, and “shell” may be a layer surrounding a part or an entirety of the core layer. For example, components constituting the shell (e.g., an inorganic powder) may be partially attached without completely covering the entire surface of the core layer. For instance, the core-shell particles may include a form in which components constituting the shell (e.g., an inorganic powder) are dispersed and attached to the core layer (e.g., silica particles). The core-shell particles may also be expressed as ‘core@shell’. For instance, core-shell particles in which a core is SiO₂ particles and a shell includes ZnO powder may also be expressed as SiO₂@ZnO.

In the “core-shell particles” of the present disclosure, a material included in the core and a material included in the shell may be bonded to each other by a natural polymer binder. In the present disclosure, the natural polymer binder may be located on a surface of a material included in the core (e.g., silica) and/or a surface of a material included in the shell (e.g., an inorganic powder). In one embodiment, the natural polymer binder may be located on a surface of a material included in the core (e.g., silica) and a surface of a material included in the shell (e.g., an inorganic powder), and a core-shell structure may be formed by bonding between the natural polymers located on the surfaces thereof. In the present disclosure, the natural polymer binder may be located inside the core and/or the shell.

In the present disclosure, the term “inorganic powder” may mean an aggregate of particles composed of an inorganic material. The inorganic powder may be one including one or a plurality of inorganic material particles. In the present disclosure, the inorganic powder may have various forms such as a spherical shape, a rod shape, a plate shape, and the like, and the form is not limited.

In the present disclosure, the term “surface modification” may mean changing a surface of a specific material (e.g., silica particles, an inorganic powder), and for instance, it may be changing chemical and/or physical characteristics of the surface of the specific material (e.g., silica particles, an inorganic powder). In the present disclosure, the surface modification may be performed through a process of mixing a first material (e.g., silica particles, an inorganic powder) with a second material (e.g., a natural polymer).

In the present disclosure, the term “wetted” inorganic powder means a state in which the inorganic powder is wet by an arbitrary material (e.g., water and/or a polyol) or a state in which the inorganic powder is well dispersed in the arbitrary material, and the form is not limited. For example, an inorganic powder wetted with an arbitrary first component (A) may be one in which the inorganic powder is mixed with the arbitrary first component (A) to become a wet state.

In the present disclosure, the term “compound including a vicinal diol” means a compound in which a hydroxyl group (—OH) is bonded to each of two adjacent atoms (e.g., carbon atoms). Two hydroxyl groups included in the compound including a vicinal diol occupy vicinal positions. That is, the two hydroxyl groups are bonded to the adjacent atoms (e.g., carbon atoms). For example, the compound including a vicinal diol may further include an additional hydroxyl group, in addition to the two hydroxyl groups bonded to the adjacent atoms (e.g., carbon atoms). In this case, the compound including a vicinal diol may be a polyol. For example, the compound including a vicinal diol may not further include an additional hydroxyl group, in addition to the two hydroxyl groups bonded to the adjacent atoms (e.g., carbon atoms). In this case, the compound including a vicinal diol may be a diol.

### Core-Shell Particles

One aspect of the present disclosure provides core-shell particles including a core including silica (SiO₂) particles; and a shell including an inorganic powder.

In one embodiment, the core-shell particles may include a natural polymer binder for bonding the silica particles and the inorganic powder. In one embodiment, the natural polymer binder may be at least one selected from the group consisting of hyaluronic acid and starch. The inorganic powder may be coated by being dispersed on a surface of the silica particles using the natural polymer binder.

In the present disclosure, “bonding” may include physical bonding or chemical bonding, and may include both direct bonding or indirect bonding.

In the present disclosure, the silica particles may also be referred to as silicon dioxide.

One aspect of the present disclosure provides core-shell particles including a core including silica (SiO₂) particles; and a shell including an inorganic powder; and including a natural polymer binder for bonding the silica particles and the inorganic powder, wherein the natural polymer binder is at least one selected from the group consisting of hyaluronic acid and starch.

In one embodiment, the core may be composed of silica particles. In one embodiment, the core-shell particles may be in a form in which an inorganic powder is coated on at least a part of a surface of the silica particles.

In one embodiment, the inorganic powder may be at least one of zinc oxide (ZnO) or titanium dioxide (TiO₂). For instance, the inorganic powder may include at least one of a plurality of zinc oxide (ZnO) particles; or a plurality of titanium dioxide (TiO₂) particles. In one embodiment, the inorganic powder may be at least one of zinc oxide (ZnO) coated with an alkyl silane or titanium dioxide (TiO₂) coated with an alkyl silane. In one embodiment, the alkyl silane may be triethoxycaprylyl silane.

In one embodiment, a diameter of the silica particles may be larger than an average diameter (or a median value of diameters) of the inorganic powder included in the shell.

In one embodiment, the diameter of the silica particles may be 100 nm to 10 m. For example, the diameter of the silica particles may be 100 nm or more, 110 nm or more, 120 nm or more, 130 nm or more, 140 nm or more, 150 nm or more, 160 nm or more, 170 nm or more, 180 nm or more, 190 nm or more, 200 nm or more, 210 nm or more, 220 nm or more, 230 nm or more, 240 nm or more, 250 nm or more, 260 nm or more, 270 nm or more, 280 nm or more, 290 nm or more, 300 nm or more, 310 nm or more, 320 nm or more, 330 nm or more, 340 nm or more, 350 nm or more, 360 nm or more, 370 nm or more, 380 nm or more, 390 nm or more, 400 nm or more, 410 nm or more, 420 nm or more, 430 nm or more, 440 nm or more, 450 nm or more, 460 nm or more, 470 nm or more, 480 nm or more, 490 nm or more, 500 nm or more, 600 nm or more, 700 nm or more, 800 nm or more, 900 nm or more, 1000 nm or more, 2 m or more, 3 m or more, 4 m or more, 5 m or more, 6 m or more, 7 m or more, 8 m or more, 9 m or more, 10 m or more, 100 nm or less, 110 nm or less, 120 nm or less, 130 nm or less, 140 nm or less, 150 nm or less, 160 nm or less, 170 nm or less, 180 nm or less, 190 nm or less, 200 nm or less, 210 nm or less, 220 nm or less, 230 nm or less, 240 nm or less, 250 nm or less, 260 nm or less, 270 nm or less, 280 nm or less, 290 nm or less, 300 nm or less, 310 nm or less, 320 nm or less, 330 nm or less, 340 nm or less, 350 nm or less, 360 nm or less, 370 nm or less, 380 nm or less, 390 nm or less, 400 nm or less, 410 nm or less, 420 nm or less, 430 nm or less, 440 nm or less, 450 nm or less, 460 nm or less, 470 nm or less, 480 nm or less, 490 nm or less, 500 nm or less, 600 nm or less, 700 nm or less, 800 nm or less, 900 nm or less, 1000 nm or less, 2 m or less, 3 m or less, 4 m or less, 5 m or less, 6 m or less, 7 m or less, 8 m or less, 9 m or less, 10 m or less, and a combination range thereof.

In one embodiment, a D₅₀ value (or median particle size) of the inorganic powder included in the shell may be 30 nm to 100 nm. For example, the D₅₀ value (or median particle size) of the inorganic powder included in the shell may be 10 nm or more, 20 nm or more, 30 nm or more, 35 nm or more, 40 nm or more, 45 nm or more, 50 nm or more, 55 nm or more, 60 nm or more, 65 nm or more, 70 nm or more, 75 nm or more, 80 nm or more, 85 nm or more, 90 nm or more, 95 nm or more, 100 nm or more, 10 nm or less, 20 nm or less, 30 nm or less, 35 nm or less, 40 nm or less, 45 nm or less, 50 nm or less, 55 nm or less, 60 nm or less, 65 nm or less, 70 nm or less, 75 nm or less, 80 nm or less, 85 nm or less, 90 nm or less, 95 nm or less, 100 nm or less, and a combination range thereof.

In one embodiment, the silica particles and the inorganic powder may each independently have surfaces thereof surface-modified with a natural polymer binder.

For example, the silica particles may be surface-modified with a first natural polymer, and the inorganic powder may be surface-modified with a second natural polymer, and in this case, the first natural polymer and the second natural polymer may be the same or different. In one embodiment, the first natural polymer and the second natural polymer may each independently be at least one selected from the group consisting of hyaluronic acid and starch. In one embodiment, the hyaluronic acid and the starch may each independently be high-pressure treated (e.g., treated with a pressure of 50 to 200 bar) or enzyme treated. In one embodiment, the first natural polymer and the second natural polymer may be bonded to form a core-shell structure.

In one embodiment, the hyaluronic acid and the starch may each independently be i) high-pressure treated, or ii) enzyme treated. In one embodiment, the hyaluronic acid may be i) high-pressure treated, or ii) enzyme treated. In one embodiment, the hyaluronic acid may be high-pressure treated. In one embodiment, the starch may be i) high-pressure treated, or ii) enzyme treated. In one embodiment, the starch may be enzyme treated.

In one embodiment, the high-pressure treatment may be applying a predetermined pressure to a sample to be treated. In one embodiment, the high-pressure treatment may be performed at room temperature.

In one embodiment, the predetermined pressure may be 50 bar to 200 bar. For example, the predetermined pressure may be 50 bar or more, 55 bar or more, 60 bar or more, 65 bar or more, 70 bar or more, 75 bar or more, 80 bar or more, 85 bar or more, 90 bar or more, 95 bar or more, 100 bar or more, 110 bar or more, 120 bar or more, 130 bar or more, 140 bar or more, 150 bar or more, 160 bar or more, 170 bar or more, 180 bar or more, 190 bar or more, 200 bar or more, 50 bar or less, 55 bar or less, 60 bar or less, 65 bar or less, 70 bar or less, 75 bar or less, 80 bar or less, 85 bar or less, 90 bar or less, 95 bar or less, 100 bar or less, 110 bar or less, 120 bar or less, 130 bar or less, 140 bar or less, 150 bar or less, 160 bar or less, 170 bar or less, 180 bar or less, 190 bar or less, 200 bar or less, or a combination range thereof.

In one embodiment, the predetermined pressure may be 1 to 1000 MPa, but is not limited thereto. For example, the predetermined pressure may be 1 MPa or more, 2 MPa or more, 3 MPa or more, 4 MPa or more, 5 MPa or more, 6 MPa or more, 7 MPa or more, 8 MPa or more, 9 MPa or more, 10 MPa or more, 20 MPa or more, 30 MPa or more, 40 MPa or more, 50 MPa or more, 60 MPa or more, 70 MPa or more, 80 MPa or more, 90 MPa or more, 100 MPa or more, 110 MPa or more, 150 MPa or more, 200 MPa or more, 250 MPa or more, 300 MPa or more, 350 MPa or more, 400 MPa or more, 450 MPa or more, 500 MPa or more, 550 MPa or more, 600 MPa or more, 650 MPa or more, 700 MPa or more, 750 MPa or more, 800 MPa or more, 850 MPa or more, 900 MPa or more, 950 MPa or more, 1000 MPa or more, 1 MPa or less, 2 MPa or less, 3 MPa or less, 4 MPa or less, 5 MPa or less, 6 MPa or less, 7 MPa or less, 8 MPa or less, 9 MPa or less, 10 MPa or less, 20 MPa or less, 30 MPa or less, 40 MPa or less, 50 MPa or less, 60 MPa or less, 70 MPa or less, 80 MPa or less, 90 MPa or less, 100 MPa or less, 110 MPa or less, 150 MPa or less, 200 MPa or less, 250 MPa or less, 300 MPa or less, 350 MPa or less, 400 MPa or less, 450 MPa or less, 500 MPa or less, 550 MPa or less, 600 MPa or less, 650 MPa or less, 700 MPa or less, 750 MPa or less, 800 MPa or less, 850 MPa or less, 900 MPa or less, 950 MPa or less, 1000 MPa or less, or a combination range thereof.

In one embodiment, the high-pressure treatment may be performed with high-pressure treatment equipment, but is not limited thereto. In one embodiment, the high-pressure treatment may be performed by injecting a sample into high-pressure treatment equipment (e.g., MF, ACC, HPH, UHPH).

In one embodiment, the high-pressure treatment equipment may be at least one selected from the group consisting of MF (Microfluidizer), ACC (Aqueous Counter Collision), HPH (High-Pressure Homogenizer), and UHPH (Ultra-High Pressure Homogenization), but is not limited thereto. The high-pressure treatment equipment is sufficient if it can apply a predetermined pressure to a sample to be treated (e.g., hyaluronic acid, starch), and a specific type is not limited.

In one embodiment, the high-pressure treatment may be a method of injecting a sample into high-pressure treatment equipment (e.g., MF, ACC, HPH, UHPH) and applying a repetitive high-pressure cycle.

In one embodiment, the high-pressure treatment may be repeatedly treating a sample (e.g., a composition including hyaluronic acid, a composition including starch) with a predetermined pressure (e.g., 50 to 200 bar) for a predetermined number of times (e.g., 1 to 30 pass, 5 to 20 pass, 10 to 20 pass, or 14 to 16 pass).

In one embodiment, the high-pressure treatment may be inputting a sample (e.g., a composition including hyaluronic acid, a composition including starch) into high-pressure treatment equipment and applying a predetermined pressure (e.g., 50 to 200 bar) to the sample.

In one embodiment, the high-pressure treatment may be inputting a sample (e.g., a composition including hyaluronic acid, a composition including starch) into high-pressure treatment equipment and repeatedly (e.g., 1 to 30 pass, 5 to 20 pass, 10 to 20 pass, or 14 to 16 pass) applying a predetermined pressure (e.g., 50 to 200 bar) to the sample.

In one embodiment, a sample used in the high-pressure treatment process may be a solution obtained by mixing hyaluronic acid and/or starch in water (e.g., distilled water) with a predetermined content (e.g., 1 to 10 wt %, 2 to 8 wt %, 2 to 6 wt %, 3 to 5 wt %, or 4 wt %) and stirring the same.

In one embodiment, an enzyme used for the enzyme treatment may be alpha-amylase (α-amylase), but is not limited thereto.

In one embodiment, the enzyme treatment may be performed by a method including steps of gelatinizing a sample to be treated (e.g., hyaluronic acid, starch) at a predetermined temperature and then reacting the same by inputting an enzyme; and cooling and drying the sample for which the enzyme reaction is completed.

In one embodiment, a gelatinization temperature in the enzyme treatment process may be 50 °C to 100 °C. For instance, the gelatinization temperature in the enzyme treatment process may be 50 °C or more, 60 °C or more, 70 °C or more, 75 °C or more, 80 °C or more, 85 °C or more, 90 °C or more, 100 °C or more, 50 °C or less, 60 °C or less, 70 °C or less, 75 °C or less, 80 °C or less, 85 °C or less, 90 °C or less, 100 °C or less, or a combination range thereof.

In the present disclosure, hyaluronic acid was written as a concept including not only hyaluronic acid itself but also a salt form of hyaluronic acid.

In one embodiment, the hyaluronic acid may be sodium hyaluronate, but is not limited thereto.

In one embodiment, the starch may be amylopectin, but is not limited thereto.

In one embodiment, the inorganic powder may be wetted with a compound including a vicinal diol. An inorganic powder surface-modified with a natural polymer may be obtained through a process of mixing the inorganic powder with a compound including a vicinal diol and the natural polymer. It is difficult to modify a surface of an inorganic powder with a natural polymer (e.g., hyaluronic acid, starch) due to hydrophobic characteristics of the inorganic powder, but a bonding force between the natural polymer and the inorganic powder may be increased by wetting the inorganic powder with a compound including a vicinal diol. An inorganic powder wetted with a compound other than the compound including a vicinal diol (e.g., a diol other than a vicinal diol) may have a poor surface modification effect even when mixed with a natural polymer.

In one embodiment, the compound including a vicinal diol may be at least one selected from the group consisting of 2,3-butylene glycol, 1,2-propanediol, 1,2-butanediol, 1,2-pentanediol, and 1,2-hexanediol, but is not limited thereto.

In one embodiment, based on a total weight of the core-shell particles, the silica particles may be included in an amount of 1 to 25 wt %, but is not limited thereto. For example, based on the total weight of the core-shell particles, the silica particles may be included in an amount of 1 wt % or more, 5 wt % or more, 10 wt % or more, 11 wt % or more, 12 wt % or more, 13 wt % or more, 15 wt % or more, 20 wt % or more, 25 wt % or more, 1 wt % or less, 5 wt % or less, 10 wt % or less, 11 wt % or less, 12 wt % or less, 13 wt % or less, 15 wt % or less, 20 wt % or less, 25 wt % or less, or a combination range thereof, but is not limited thereto.

In one embodiment, based on the total weight of the core-shell particles, the inorganic powder may be included in an amount of 1 to 60 wt %, but is not limited thereto. For example, based on the total weight of the core-shell particles, the inorganic powder may be included in an amount of 1 wt % or more, 5 wt % or more, 10 wt % or more, 15 wt % or more, 20 wt % or more, 25 wt % or more, 30 wt % or more, 35 wt % or more, 36 wt % or more, 37 wt % or more, 38 wt % or more, 39 wt % or more, 40 wt % or more, 45 wt % or more, 50 wt % or more, 55 wt % or more, 60 wt % or more, 1 wt % or less, 5 wt % or less, 10 wt % or less, 15 wt % or less, 20 wt % or less, 25 wt % or less, 30 wt % or less, 35 wt % or less, 36 wt % or less, 37 wt % or less, 38 wt % or less, 39 wt % or less, 40 wt % or less, 45 wt % or less, 50 wt % or less, 55 wt % or less, 60 wt % or less, or a combination range thereof, but is not limited thereto.

In one embodiment, within the core-shell particles, a weight ratio of the silica particles and the inorganic powder may be 1:1 to 1:10, 1:2 to 1:10, 1:2.5 to 1:10, 1:3 to 1:10, 1:2.5 to 1:9, 1:2.5 to 1:8, 1:2.5 to 1:7, 1:2.5 to 1:6, 1:2.5 to 1:5.5, 1:2.5 to 1:5, 1:2.5 to 1:4.5, 1:3 to 1:4.5, or 1:2.5 to 1:3.5.

In addition, one aspect of the present disclosure may provide core-shell particles as core-shell particles including a core and a shell, wherein the core includes silica particles surface-modified with a first natural polymer, the shell includes an inorganic powder surface-modified with a second natural polymer, the first natural polymer and the second natural polymer are each independently at least one selected from the group consisting of hyaluronic acid and starch, and the first natural polymer and the second natural polymer are bonded.

In one embodiment, the hyaluronic acid and the starch may each independently be high-pressure treated or enzyme treated.

Since hyaluronic acid, starch, high-pressure treatment, enzyme treatment, silica particles, inorganic powder, and the like have been described above, specific descriptions thereof are omitted.

### Composition for Ultraviolet Blocking Including Core-Shell Particles

One aspect of the present disclosure provides a composition for ultraviolet blocking including core-shell particles.

Since the core-shell particles are the same as described above, a specific description thereof is omitted.

In one embodiment, the composition for ultraviolet blocking may be an inorganic composition for ultraviolet blocking.

In one embodiment, the composition for ultraviolet blocking may be a cosmetic composition, a food composition, an oral composition, a non-therapeutic composition, a non-therapeutic oral composition, a pharmaceutical composition, or a composition for external application. Specifically, the composition for ultraviolet blocking may be a composition for external application or a cosmetic composition, but is not limited thereto.

In one embodiment, the composition for ultraviolet blocking may be a water-dispersion formulation, but is not limited thereto. Since the core-shell particles are in a form in which an inorganic powder representing hydrophobic characteristics is effectively dispersed, a composition for ultraviolet blocking of a water-dispersion formulation may be implemented by utilizing such core-shell particles.

In one embodiment, the composition for ultraviolet blocking may be composed of OTC (Over-the-Counter) raw materials so that there is no need to receive additional permission for commercialization.

In one embodiment, the composition for ultraviolet blocking may be treated one time or a plurality of times on a subject in need of ultraviolet blocking, but is not limited thereto.

In one embodiment, the composition for ultraviolet blocking may be treated in combination with any other component capable of representing an ultraviolet blocking effect on a subject in need of ultraviolet blocking, but is not limited thereto.

In one embodiment, the composition for ultraviolet blocking may further include any other component capable of representing an ultraviolet blocking effect.

In one embodiment, the composition for ultraviolet blocking may further include components that may be included in cosmetics. For example, the composition for ultraviolet blocking may further include additives such as a preservative, a thickener, a viscosity modifier, a stabilizer, a pearling agent, a metal ion sequestering agent, a cationic surfactant, a pH modifier, a fragrance, and a dye, and these may be easily purchased and used commercially.

In one embodiment, the composition for ultraviolet blocking may be commercialized in the form of a toner, a lotion, a cream, a serum, an essence, an eye cream, a body lotion, a body cream, a powder, an ointment, a spray, or a concealer stick, but is not limited thereto.

The composition for ultraviolet blocking including core-shell particles according to one aspect of the present disclosure may represent an ultraviolet blocking effect superior to a composition in which a component included in a core (e.g., silica particles) and an inorganic powder included in a shell are simply mixed (mixed identically to a content included in the core-shell particles).

Method for Ultraviolet Blocking (or Method for Protecting Skin of Subject from Ultraviolet Rays)

One aspect of the present disclosure provides a method for ultraviolet blocking including a step of applying a composition including core-shell particles to a subject. For example, the step of applying the composition including core-shell particles to the subject may be a step of applying the core-shell particles to a surface (or skin) of the subject.

One aspect of the present disclosure provides a method for protecting skin of a subject from ultraviolet rays including a step of applying a composition including core-shell particles to the subject. For example, the step of applying the composition including core-shell particles to the subject may be a step of applying the core-shell particles to a surface (or skin) of the subject.

Since the core-shell particles and the composition including the core-shell particles are the same as described above, a specific description thereof is omitted.

In one embodiment, the subject may be an animal including a mammal (e.g., a human), but is not limited thereto.

In one embodiment, the subject may be a subject in need of ultraviolet blocking.

In the present disclosure, the applying may include administration or application to skin. For example, the administration may be administration through oral administration, transdermal administration, subcutaneous administration, intravenous administration, intraperitoneal administration, intramuscular administration, application, or topical application, but is not limited thereto.

In one embodiment, a daily application amount of the core-shell particles or the composition including the core-shell particles may be 0.0001 to 10000 mg/kg, but is not limited thereto. It should be understood that the application amount of the core-shell particles or the composition including the core-shell particles is to be determined in light of various related factors such as an administration route, an age, a gender, and a weight of a subject, and therefore, the application amount does not limit a scope of the present invention in any way.

In one embodiment, the core-shell particles or the composition including the core-shell particles may be applied to a subject once a day or divided into several times, but is not limited thereto.

In one embodiment, the core-shell particles or the composition including the core-shell particles may be applied in combination with any other component capable of representing a skin improvement effect and/or an ultraviolet blocking effect. A specific type of the any other component is not limited.

### Use of Core-Shell Particles

One aspect of the present disclosure provides a use of core-shell particles.

One aspect of the present disclosure provides a use of core-shell particles for manufacture of a composition for ultraviolet blocking. One aspect of the present disclosure provides a use of a composition including core-shell particles for manufacture of a composition for ultraviolet blocking.

One aspect of the present disclosure provides a use of core-shell particles for ultraviolet blocking. One aspect of the present disclosure provides a use of a composition including core-shell particles for ultraviolet blocking.

Since the core-shell particles and the composition for ultraviolet blocking are the same as described above, specific descriptions thereof are omitted.

### Method for Manufacturing Core-Shell Particles

One aspect of the present disclosure provides a method for manufacturing core-shell particles. Since the core-shell particles have been described above, a specific description thereof is omitted.

In one embodiment, the method for manufacturing core-shell particles may include steps of: (a) manufacturing silica particles surface-modified with a first natural polymer; (b) manufacturing an inorganic powder surface-modified with a second natural polymer; and (c) manufacturing core-shell particles by mixing the silica particles surface-modified with the first natural polymer and the inorganic powder surface-modified with the second natural polymer.

In one embodiment, the first natural polymer and the second natural polymer may each independently be at least one selected from the group consisting of hyaluronic acid and starch. In one embodiment, the hyaluronic acid and the starch may each independently be i) high-pressure treated, or ii) enzyme treated.

In one embodiment, the first natural polymer and the second natural polymer may each independently be at least one selected from the group consisting of i) high-pressure treated or enzyme treated hyaluronic acid and ii) high-pressure treated or enzyme treated starch.

Since the silica particles, the inorganic powder, the hyaluronic acid, the starch, the high-pressure treatment, the high-pressure treatment equipment, the enzyme treatment, and the like have been described above, specific descriptions thereof are omitted.

When surfaces of silica particles and an inorganic powder are surface-modified with a high-pressure treated or enzyme treated natural polymer, respectively, and then mixed, a core-shell structure may be formed by a simple process without involving an additional chemical reaction.

Meanwhile, when surfaces of core particles and a surface of an inorganic powder are modified with a natural polymer which is not high-pressure treated (e.g., hyaluronic acid which is not high-pressure treated using high-pressure treatment equipment) and then mixed, a coating density of a shell layer including the inorganic powder becomes very low and aggregation between shell particles may occur more than aggregation between a core and a shell. As a result, in a case of using a natural polymer which is not high-pressure treated or enzyme treated (e.g., hyaluronic acid which is not high-pressure treated using high-pressure treatment equipment, starch which is not enzyme treated), a surface of the core particles is mostly exposed and inorganic powders become aggregated with each other, and as a result, the core-shell structure may not be well formed. In addition, unlike an enzyme treated natural polymer, a natural polymer which is not enzyme treated (e.g., starch which is not enzyme treated) is not well dissolved/dispersed in water, and as a result, it may not be easy to modify a surface of core particles or an inorganic powder.

In one embodiment, step (a) may be a step of manufacturing silica particles surface-modified with a first natural polymer by mixing the silica particles with a dispersion in which the first natural polymer is dispersed.

In one embodiment, the dispersion in which the first natural polymer is dispersed may be one in which at least one selected from the group consisting of i) high-pressure treated or enzyme treated hyaluronic acid and ii) high-pressure treated or enzyme treated starch is dispersed in water.

In one embodiment, in a mixture in which the silica particles and the dispersion in which the first natural polymer is dispersed are mixed, a content of the silica particles may be 30 to 70 wt %, 40 to 60 wt %, or 45 to 55 wt % based on a total weight of the mixture, but is not limited thereto.

In one embodiment, step (b) may be a step of manufacturing an inorganic powder surface-modified with a second natural polymer by mixing the inorganic powder with a dispersion in which the second natural polymer is dispersed. In one embodiment, step (b) may be a step of manufacturing an inorganic powder surface-modified with a second natural polymer by mixing an inorganic powder coated with an alkyl silane (e.g., ZnO powder coated with an alkyl silane) with a dispersion in which the second natural polymer is dispersed.

In one embodiment, step (b) may be a step of manufacturing an inorganic powder surface-modified with a second natural polymer by mixing the inorganic powder with a dispersion in which the second natural polymer is dispersed and a compound including a vicinal diol. In one embodiment, step (b) may be a step of manufacturing an inorganic powder surface-modified with a second natural polymer by mixing an inorganic powder coated with an alkyl silane (e.g., ZnO powder coated with an alkyl silane) with a dispersion in which the second natural polymer is dispersed and a compound including a vicinal diol.

In one embodiment, the compound including a vicinal diol may be at least one selected from the group consisting of 2,3-butylene glycol, 1,2-propanediol, 1,2-butanediol, 1,2-pentanediol, and 1,2-hexanediol.

In one embodiment, the dispersion in which the second natural polymer is dispersed may be one in which at least one selected from the group consisting of i) high-pressure treated or enzyme treated hyaluronic acid and ii) high-pressure treated or enzyme treated starch is dispersed in water.

In one embodiment, in a mixture in which the inorganic powder and the dispersion in which the second natural polymer is dispersed (or the dispersion in which the second natural polymer is dispersed and the compound including a vicinal diol) are mixed, a content of the inorganic powder may be 30 to 70 wt %, 40 to 60 wt %, or 45 to 55 wt % based on a total weight of the mixture, but is not limited thereto.

In one embodiment, an order of step (a) and step (b) is not limited. For example, step (a) and step (b) may be performed simultaneously. For another example, step (a) may be performed before step (b). For another example, step (b) may be performed before step (a).

A conventional core-shell manufacturing method by synthesis using a chemical substance includes several steps, and precise control of conditions under which a reaction is performed in each step is required. Meanwhile, the method for manufacturing core-shell particles according to one aspect of the present disclosure has advantages that a process is simple, generation of by-products is small, a yield is high, and it is eco-friendly. In addition, the core-shell particles manufactured by the manufacturing method according to one aspect of the present disclosure may minimize skin irritation because a natural polymer is used as a binder for bonding a core and a shell instead of other chemical substances.

Hereinafter, the present invention will be described in detail by giving Examples in order to specifically describe the present invention. The following Examples are only provided for the purpose of illustration to help understanding of the present invention, and a category and a scope of the present invention are not limited thereby.

### 1. Manufacture of Examples: Manufacture of Core-Shell Particles

### (1) Example 1: SiO₂@ZnO Manufactured by Using High-Pressure Treated Natural Polymer as Binder

### 1) Preparation of Surface-Modified Core Component

SiO₂ nanoparticles (Moonsil Naver 300, INCI: SILICA) were mixed with a natural polymer dispersed in water to manufacture a mixture including surface-modified SiO₂ nanoparticles. Based on a total weight of the mixture, SiO₂ was 50 wt %, the natural polymer was 1 wt %, and water was 49 wt %. Here, the natural polymer used is hyaluronic acid high-pressure treated with a pressure of 100 bar by using MF (Microfluidizer) equipment. The hyaluronic acid used in Examples/Comparative Examples of the present disclosure is sodium hyaluronate. That is, the high-pressure treated hyaluronic acid is high-pressure treated sodium hyaluronate.

### 2) Preparation of Surface-Modified Shell Component

A hydrophobic ZnO inorganic powder coated with an alkyl silane (MZX-508OTS, INCI: Zinc oxide, Triethoxycaprylyl silane) was mixed with a natural polymer dispersed in water and a polyol including a vicinal diol to manufacture a mixture including a surface-modified inorganic powder. Here, the natural polymer used is hyaluronic acid high-pressure treated with MF equipment. The hyaluronic acid high-pressure treated with MF equipment was manufactured by the same method as the method manufactured in 1) above. Based on the total weight of the mixture, the hydrophobic ZnO inorganic powder was 50 wt %, the natural polymer was 1 wt %, water was 24 wt %, and the polyol was 25 wt %. 2,3-butylene glycol was used as the polyol, but it may be replaced with a compound including adjacent —OH groups such as 1,2-propanediol, 1,2-butanediol, 1,2-pentanediol, and 1,2-hexanediol.

### 3) Mixing of Core Component and Shell Component

The mixture including surface-modified SiO₂ nanoparticles and the mixture including surface-modified inorganic powder prepared above were mixed in a ratio of 1:3 for 2 hours by using a paddle mixer to manufacture a SiO₂@ZnO core-shell structure. Based on a total weight of the core-shell structure, SiO₂ was 12.5 wt %, the ZnO inorganic powder was 37.5 wt %, the natural polymer was 1 wt %, water was 18.75 wt %, and the polyol was 30.25 wt %.

As a result of confirming core-shell bonding strength through ultrasonic treatment of 10% solute, it was shown that inorganic powder particles were stably bonded to a surface of core particles. The high-pressure treated natural polymer, as a binder, could improve a bonding force between the SiO₂ core and the inorganic powder particles included in the shell.

### (2) Example 2: SiO₂@TiO₂ Manufactured by Using High-Pressure Treated Natural Polymer as Binder

### 1) Preparation of Surface-Modified Core Component

SiO₂ nanoparticles (Moonsil Naver 300, INCI: SILICA) were mixed with a natural polymer dispersed in water to manufacture a mixture including surface-modified SiO₂ nanoparticles. Based on a total weight of the mixture, SiO₂ was 50 wt %, the natural polymer was 1 wt %, and water was 49 wt %. Here, the natural polymer used during manufacture of Example 2 is hyaluronic acid high-pressure treated with a pressure of 100 bar by using MF equipment.

### 2) Preparation of Surface-Modified Shell Component

A TiO₂ inorganic powder (Micro TiO₂ 060 AS, INCI: TITANIUM DIOXIDE (NANO)*TRIETHOXYCAPRYLYL SILANE*ALUMINUM HYDROXIDE) was mixed with a natural polymer dispersed in water and a polyol including a vicinal diol to manufacture a mixture including a surface-modified inorganic powder. Here, the natural polymer used is hyaluronic acid high-pressure treated by using MF equipment. The hyaluronic acid high-pressure treated by using MF equipment was manufactured by the same method as the method manufactured in 1) above. Based on the total weight of the mixture, the TiO₂ inorganic powder was 50 wt %, the natural polymer was 1 wt %, water was 24 wt %, and the polyol was 25 wt %. 2,3-butylene glycol was used as the polyol, but it may be replaced with a compound including adjacent —OH groups such as 1,2-propanediol, 1,2-butanediol, 1,2-pentanediol, and 1,2-hexanediol.

### 3) Mixing of Core Component and Shell Component

The mixture including surface-modified SiO₂ nanoparticles and the mixture including surface-modified inorganic powder prepared above were mixed in a ratio of 1:3 for 2 hours by using a paddle mixer to manufacture a SiO₂@TiO₂ core-shell structure. Based on a total weight of the core-shell structure, SiO₂ was 12.5 wt %, the TiO₂ inorganic powder was 37.5 wt %, the natural polymer was 1 wt %, water was 18.75 wt %, and the polyol was 30.25 wt %.

As a result of confirming core-shell bonding strength through ultrasonic treatment of 10% solute, it was shown that inorganic powder particles were stably bonded to a surface of core particles.

### (3) Example 3: SiO₂@ZnO Manufactured by Using High-Pressure Treated Natural Polymer as Binder

It was manufactured by the same method as Example 1, but enzyme treated starch was used as the natural polymer instead of hyaluronic acid high-pressure treated by using MF equipment. The enzyme treated starch was manufactured by the following method: after gelatinizing starch under a condition of 75 to 85 °C, an enzyme was input and an enzyme reaction was completed within 1 hour. Thereafter, it was cooled and dried. Alpha-amylase was used as the enzyme.

### (4) Example 4: SiO₂@TiO₂ Manufactured by Using High-Pressure Treated Natural Polymer as Binder

It was manufactured by the same method as Example 2, but enzyme treated starch was used as the natural polymer instead of hyaluronic acid high-pressure treated by using MF equipment. The enzyme treated starch was manufactured by the following method: after gelatinizing starch under a condition of 75 to 85 °C, an enzyme was input and an enzyme reaction was completed within 1 hour. Thereafter, it was cooled and dried. Alpha-amylase was used as the enzyme.

### 2. Manufacture of Comparative Examples

### (1) Comparative Example 1: Composition in which SiO₂ and ZnO Inorganic Powder are Simply Mixed

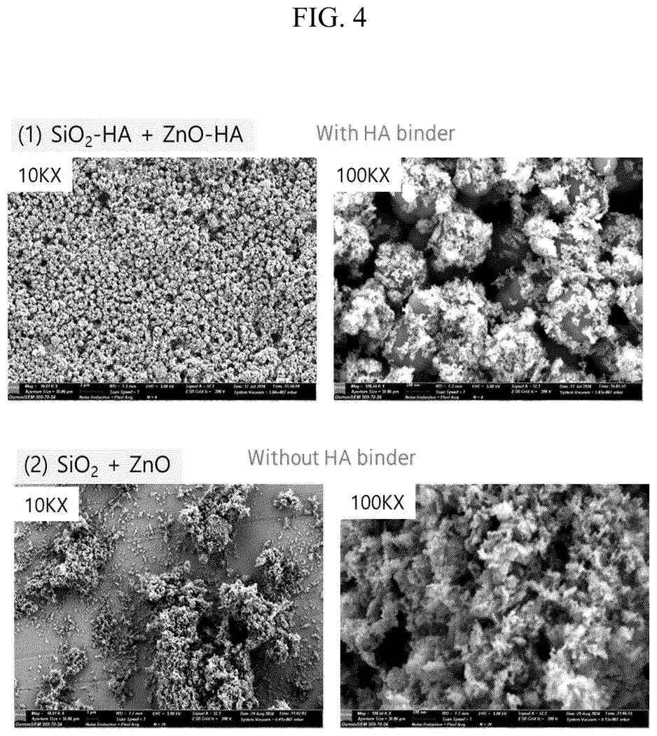

For comparison, SiO₂ and ZnO inorganic powder were simply mixed. That is, in a manufacturing process, both SiO₂ and ZnO inorganic powder were not surface-modified with a natural polymer binder. Contents of SiO₂ and ZnO in the mixture were made to be the same as Example 1. As a result, a core-shell structure of SiO₂ and ZnO was not formed, and aggregation of ZnO powder occurred (with reference to (2) of FIG. 4).

### (2) Comparative Example 2: Composition in which ZnO Inorganic Powder Surface-Modified with Natural Polymer is Mixed with SiO₂

Only ZnO inorganic powder was surface-modified with a high-pressure treated natural polymer, and then mixed with SiO₂. In a final mixture, contents of SiO₂, ZnO inorganic powder, hyaluronic acid, polyol, and water were made to be the same as Example 1. A specific manufacturing method is as follows.

### 1) Preparation of Surface-Modified ZnO

A hydrophobic ZnO inorganic powder coated with an alkyl silane (MZX-508OTS, INCI: Zinc oxide, Triethoxycaprylyl silane) was mixed with a natural polymer dispersed in water and a polyol including a vicinal diol to manufacture a mixture including a surface-modified inorganic powder. The natural polymer used here is a natural polymer high-pressure treated identically to that used for ZnO surface modification of Example 1. Based on the total weight of the mixture, the hydrophobic ZnO inorganic powder was 50 wt %, the natural polymer was 1 wt %, water was 24 wt %, and the polyol was 25 wt %. 2,3-butylene glycol was used as the polyol, but it may be replaced with a compound including adjacent —OH groups such as 1,2-propanediol, 1,2-butanediol, 1,2-pentanediol, and 1,2-hexanediol.

2) Manufacture of Mixture of SiO₂ and ZnO Inorganic Powder Surface-Modified with Natural Polymer

SiO₂ nanoparticles (Moonsil Naver 300, INCI: SILICA) were mixed with the surface-modified ZnO prepared in 1) above to obtain a mixture.

### (3) Comparative Example 3: Composition in which TiO₂ Inorganic Powder Surface-Modified with Natural Polymer is Mixed with SiO₂

Only TiO₂ inorganic powder was surface-modified with a high-pressure treated natural polymer, and then mixed with SiO₂. In a final mixture, contents of SiO₂, TiO₂ inorganic powder, hyaluronic acid, polyol, and water were made to be the same as Example 2. A specific manufacturing method is as follows.

### 1) Preparation of Surface-Modified TiO₂

A TiO₂ inorganic powder (Micro TiO₂ 060 AS, INCI: TITANIUM DIOXIDE (NANO)*TRIETHOXYCAPRYLYL SILANE*ALUMINUM HYDROXIDE) was mixed with a natural polymer dispersed in water and a polyol including a vicinal diol to manufacture a mixture including a surface-modified inorganic powder. The natural polymer used here is a natural polymer high-pressure treated identically to that used for TiO₂ surface modification of Example 2. Based on the total weight of the mixture, the TiO₂ inorganic powder was 50 wt %, the natural polymer was 1 wt %, water was 24 wt %, and the polyol was 25 wt %. 2,3-butylene glycol was used as the polyol, but it may be replaced with a compound including adjacent —OH groups such as 1,2-propanediol, 1,2-butanediol, 1,2-pentanediol, and 1,2-hexanediol.

2) Manufacture of Mixture of SiO₂ and TiO₂ Inorganic Powder Surface-Modified with Natural Polymer

SiO₂ nanoparticles (Moonsil Naver 300, INCI: SILICA) were mixed with the surface-modified TiO₂ prepared in 1) above to obtain a mixture.

### (4) Comparative Example 4: SiO₂@ZnO Manufactured by Using Natural Polymer which is not High-Pressure Treated or Enzyme Treated as Binder

It was manufactured by the same method as Example 1, but hyaluronic acid which was not high-pressure treated was used as a natural polymer (that is, general hyaluronic acid was used instead of high-pressure treated hyaluronic acid). In a final mixture, contents of SiO₂, ZnO inorganic powder, hyaluronic acid, polyol, and water were made to be the same as Example 1.

### (5) Comparative Example 5: SiO₂@TiO₂ Manufactured by Using Natural Polymer which is not High-Pressure Treated or Enzyme Treated as Binder

It was manufactured by the same method as Example 2, but hyaluronic acid which was not high-pressure treated was used as a natural polymer (that is, general hyaluronic acid was used instead of high-pressure treated hyaluronic acid). In a final mixture, contents of SiO₂, TiO₂ inorganic powder, hyaluronic acid, polyol, and water were made to be the same as Example 2.

### 3. Experimental Example 1: Structure Evaluation of Examples and Comparative Examples (Evaluation of Core-Shell Structure Formation)

The structures of the Examples and Comparative Examples were evaluated through SEM measurement. A thin film was prepared by spreading a very small drop of a sample on a silicon wafer substrate. The sample was fixed to a holder and placed in the vacuum chamber of the SEM equipment. A high vacuum was created inside the chamber, and an electron beam (3 KV) emitted from an electron gun was irradiated onto the surface of the sample. Secondary electrons and backscattered electrons emitted from the sample surface were detected to generate SEM (Scanning Electron Microscope) images.

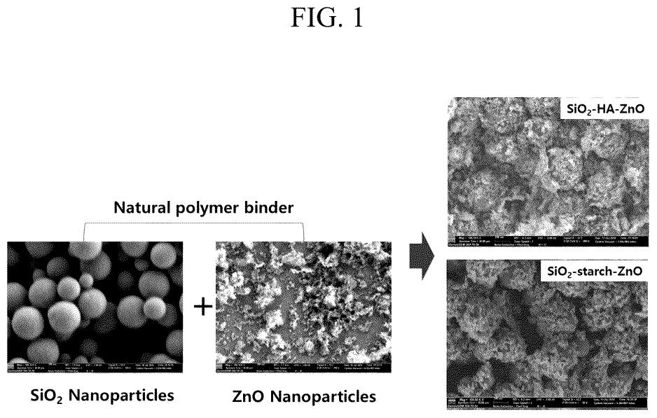

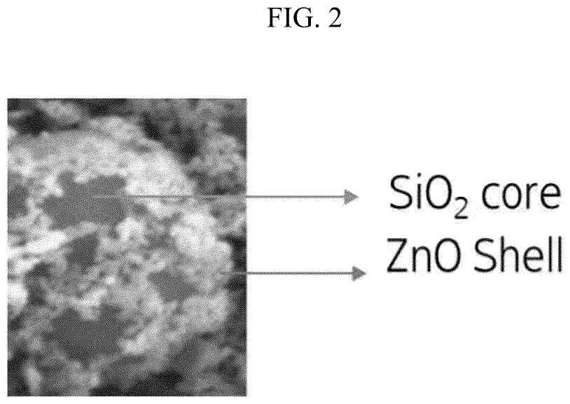

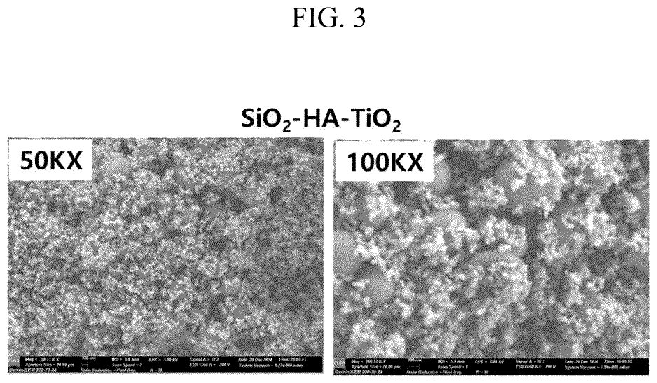

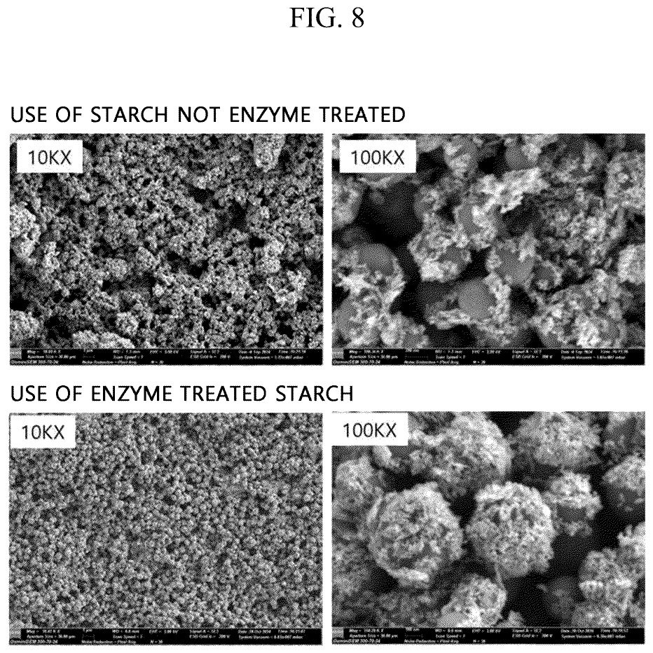

As a result of the SEM measurement, in the Examples manufactured by using a high-pressure treated or enzyme treated natural polymer as a binder, core-shell particles were well formed, and inorganic powder particles were distributed and stably bonded to the surface of the core particles (refer to FIGS. 1 to 3, (1) of FIG. 4, and the bottom photo of FIG. 8). SiO₂-HA-ZnO of FIG. 1 is an SEM image of the core-shell particles of Example 1, and SiO₂-starch-ZnO is an SEM image of the core-shell particles of Example 3. FIG. 2 is an enlarged view of a part of the SEM image of the SiO₂@ZnO core-shell particles. FIG. 3 is an SEM image of the SiO₂@TiO₂ core-shell particles of Example 2. (1) of FIG. 4 is an SEM image of the core-shell particle of Example 1. The bottom photo of FIG. 8 (using enzyme treated starch) is an SEM image of the core-shell particle of Example 3. The top two photos of FIG. 8 are SEM photos of a Comparative Example manufactured by using starch that was not enzyme treated as a binder.

On the other hand, in the case of a composition in which SiO₂ and ZnO inorganic powder were simply mixed without using a natural polymer binder (Comparative Example 1), a core-shell structure was not formed, and aggregation occurred between the ZnO inorganic powders (refer to (2) of FIG. 4). (2) of FIG. 4 is an SEM image of a composition in which SiO₂ and ZnO inorganic powder were simply mixed.

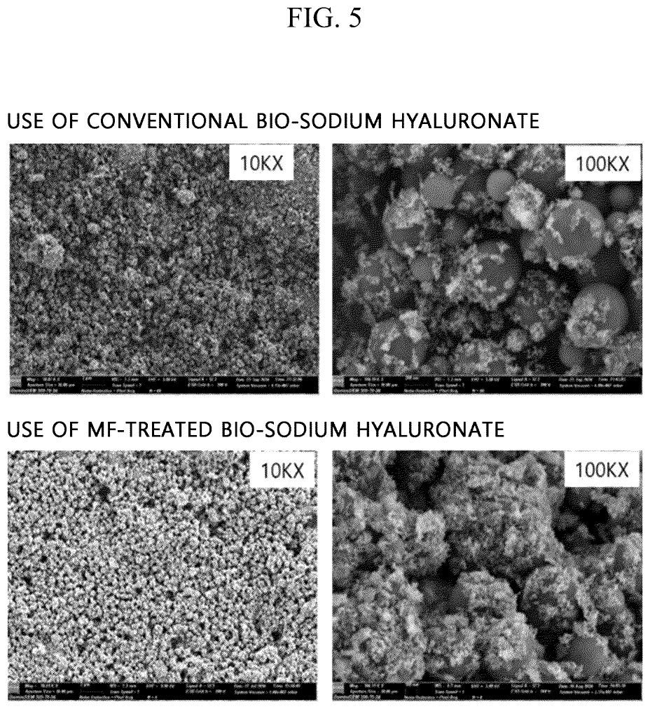

Meanwhile, in Comparative Examples (Comparative Example 4 to Comparative Example 5) manufactured by using a general natural polymer (e.g., bio-sodium hyaluronate) that was not high-pressure treated or enzyme treated as a binder, inorganic powder particles adhered to a part of the surface of the core particles, but the coating density of the shell layer was very low, and aggregation mainly occurred between the inorganic powder particles included in the shell. The top two photos of FIG. 5 are SEM photos of a Comparative Example manufactured by using a general natural polymer that was not high-pressure treated or enzyme treated as a binder, and the bottom two photos are SEM photos of a Comparative Example manufactured by using a high-pressure treated natural polymer as a binder. As can be seen in the top two photos of FIG. 5, in the case of Comparative Examples 4 to 5, the surface of the core particles was mostly exposed, and aggregates of ZnO particles were observed on a part of the surface of the core particles. For reference, in the case of starch that was not enzyme treated, unlike enzyme treated starch, it was not dissolved/dispersed in water, making comparative experiments difficult.

### 4. Experimental Example 2: Evaluation of Light Scattering Effect

The light scattering effect was evaluated through UV-Vis absorption data measurement. A sample to be measured was dispersed in an absolute ethanol solvent at a concentration of 0.05 mg/ml through ultrasonic treatment for 30 seconds. The wavelength range of the instrument was set to 200-800 nm, and absorbance was measured using a quartz cuvette. A solvent background spectrum was measured with a cuvette containing only the ethanol solvent and set as a baseline. The sample to be measured was put into the cuvette, and the cuvette was mounted in the instrument to measure the absorbance of the sample in the set wavelength range. The light scattering effect of the sample was evaluated through the measured absorbance spectrum.

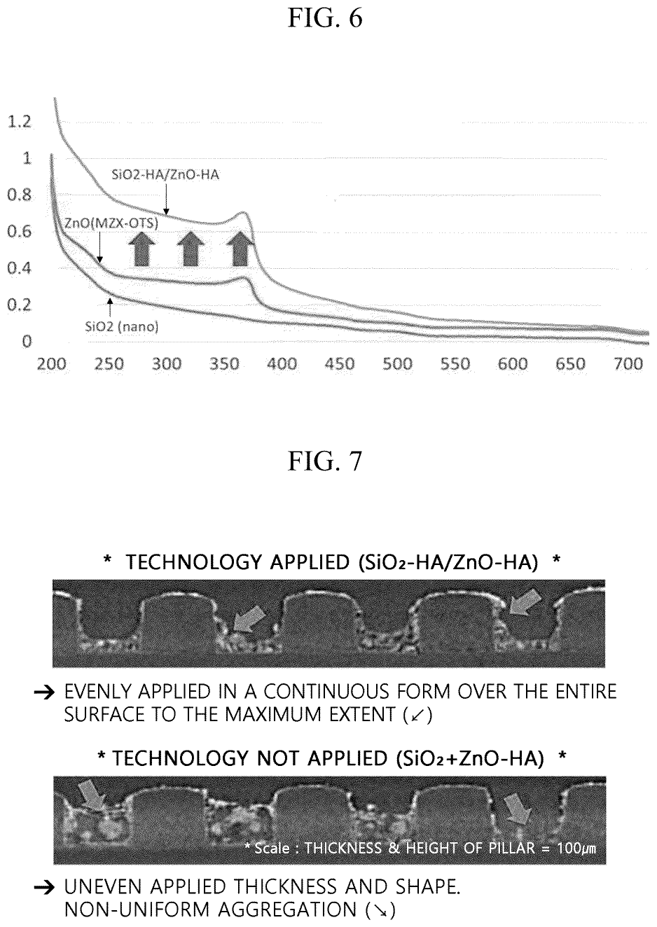

As can be seen in FIG. 6, core-shell particles (SiO₂—HA/ZnO—HA) in which inorganic powder is coated on the surface of silica particles had a light scattering effect about twice as high as that of an identical content of ZnO (ZnO(MZX—OTS)) that does not have a core-shell structure.

### 5. Experimental Example 3: Evaluation of Ultraviolet Blocking Effect

The ultraviolet blocking effect was evaluated through in-vitro SPF measurement. Specifically, in-vitro SPF was measured using a UV2000S instrument based on the FDA 2011 method. Three PMMA plates were used per sample, and 0.019 g of the sample was applied to each plate. After applying the sample, it was dried at room temperature for 15 minutes. As a control group, SPF was measured at five sites of an empty PMMA plate, and the average SPF value was calculated to set the baseline. SPF was measured at five sites on each of the three PMMA plates to which the sample was applied, and the average SPF value was calculated (measured at a total of 15 sites).

As a result, as shown in Table 1 below, the core-shell particles of Example 1, in which ZnO inorganic powder was bonded to the surface of SiO₂ using a high-pressure treated natural polymer as a binder, showed a 37% increase in in-vitro SPF compared to Comparative Example 2. In addition, the core-shell particles of Example 2, in which TiO₂ inorganic powder was bonded to the surface of SiO₂ using a high-pressure treated natural polymer as a binder, showed a 39% increase in in-vitro SPF compared to Comparative Example 3.

TABLE 1

| | SPF of Comparative | SPF increase rate of |
|---|---|---|
| SPF of Example 1 | Example 2 | Example 1 compared to |
| (SiO₂-HA/ZnO-HA) | (SiO₂ + ZnO-HA) | Comparative Example 2 |
| 15.1 | 11 | 37% |
| | SPF of Comparative | SPF increase rate of |
| SPF of Example 2 | Example 3 | Example 2 compared to |
| (SiO₂-HA/TiO₂-HA) | (SiO₂ + TiO₂-HA) | Comparative Example 3 |
| 33.5 | 24.1 | 39% |

### 6. Experimental Example 4: Evaluation of Uniform Application to Skin

The application uniformity of the Examples and Comparative Examples was evaluated using a self-developed skin mimic. Specifically, the Examples and Comparative Examples were each applied to a self-developed skin mimic under the same conditions, and the appearances thereof were confirmed. The core-shell particles of the Examples were evenly applied in a continuous form over the surface of the skin mimic to the maximum extent. On the other hand, the materials of the Comparative Examples were not evenly applied in terms of thickness and shape to the surface of the skin mimic and showed a non-uniform aggregation on the surface of the skin mimic. The top photo of FIG. 7 is a photo of the core-shell particles of Example 1 applied to a skin mimic, and the bottom photo is a photo of Comparative Example 2 applied to a skin mimic.

What is claimed is:

1. A core-shell particle, comprising:
a core comprising silica (SiO₂) particles;
a shell comprising an inorganic powder; and
a natural polymer binder for bonding the silica particles and the inorganic powder,
wherein the natural polymer binder is at least one selected from the group consisting of hyaluronic acid and starch.

2. The core-shell particle of claim 1, wherein the core is composed of silica particles.

3. The core-shell particle of claim 1, wherein the inorganic powder is at least one of zinc oxide (ZnO) or titanium dioxide (TiO₂).

4. The core-shell particle of claim 1, wherein the inorganic powder is at least one of zinc oxide (ZnO) coated with an alkyl silane or titanium dioxide (TiO₂) coated with an alkyl silane.

5. The core-shell particle of claim 1, wherein the hyaluronic acid and starch are each independently i) high-pressure treated, or ii) enzyme treated.

6. The core-shell particle of claim 5, wherein the high-pressure treatment is performed by applying a pressure of 50 to 200 bar.

7. The core-shell particle of claim 1, wherein a diameter of the silica particles is larger than an average diameter of the inorganic powder included in the shell.

8. The core-shell particle of claim 1, wherein the inorganic powder is wetted with a compound comprising a vicinal diol.

9. The core-shell particle of claim 1, wherein the silica particles are included in an amount of 1 to 25 wt % based on a total weight of the core-shell particle.

10. The core-shell particle of claim 1, wherein the inorganic powder is included in an amount of 1 to 60 wt % based on a total weight of the core-shell particle.

11. A core-shell particle, comprising a core and a shell,
wherein the core comprises silica particles surface-modified with a first natural polymer,
the shell comprises an inorganic powder surface-modified with a second natural polymer,
the first natural polymer and the second natural polymer are each independently at least one selected from the group consisting of hyaluronic acid and starch, and the first natural polymer and the second natural polymer are bonded.

12. The core-shell particle of claim 11, wherein the hyaluronic acid and starch are each independently high-pressure treated or enzyme treated.

13. A method for protecting skin of a subject from ultraviolet rays, the method comprising: applying a composition comprising the core-shell particle of claim 1 to the subject.

14. A method for manufacturing the core-shell particle of claim 1, the method comprising steps of:
(a) manufacturing silica particles surface-modified with a first natural polymer;
(b) manufacturing an inorganic powder surface-modified with a second natural polymer; and
(c) manufacturing core-shell particles by mixing the silica particles surface-modified with the first natural polymer and the inorganic powder surface-modified with the second natural polymer,
wherein the first natural polymer and the second natural polymer are each independently at least one selected from the group consisting of i) high-pressure treated or enzyme treated hyaluronic acid and ii) high-pressure treated or enzyme treated starch.

15. The method of claim 14, wherein step (a) is a step of manufacturing silica particles surface-modified with the first natural polymer by mixing silica (SiO₂) particles with a dispersion in which the first natural polymer is dispersed.

16. The method of claim 14, wherein step (b) is a step of manufacturing an inorganic powder surface-modified with the second natural polymer by mixing an inorganic powder with a dispersion in which the second natural polymer is dispersed.

17. The method of claim 14, wherein step (b) is a step of manufacturing an inorganic powder surface-modified with the second natural polymer by mixing an inorganic powder with a dispersion in which the second natural polymer is dispersed and a compound comprising a vicinal diol.

18. The method of claim 17, wherein the compound comprising a vicinal diol is at least one selected from the group consisting of 2,3-butylene glycol, 1,2-propanediol, 1,2-butanediol, 1,2-pentanediol, and 1,2-hexanediol.

19. The method of claim 14, wherein the high-pressure treatment is performed by applying a pressure of 50 to 200 bar.
